(19) United States
(12) Patent Application Publication
(10) Pub. No.: US 2002/0160421 A1
(43) Pub. Date: Oct. 31, 2002

(54) Method for monitoring and validating stress induction of disease state

(76) Inventors: George Jackowski, Kettleby (CA); Eric B. Stanton, Burlington (CA)

Correspondence Address: MCHALE & SLAVIN, 4440 PGA BLVD, SUITE 402, PALM BEACH GARDENS, FL, 33410

(21) Appl. No.: 09/846,341

(22) Filed: Apr. 30, 2001

(51) Int. Cl. C12Q 1/68; G01N 33/53; G06F 19/00; G01N 33/48; G01N 33/50

(52) U.S. Cl. 435/7.1; 435/6; 702/19; 702/20

(57) ABSTRACT

The present invention provides a biochemically-based methodology for ascertaining the presence and/or verifying the historical release of biopolymers, which have been shown to be indicative of a disease state or are predictive of the development of said disease state.

### FIELD OF THE INVENTION

This invention relates to the utilization of biochemical investigative techniques to monitor disease state, and particularly relates to the diagnosis and validation of disease states, or precursor conditions thereof, whose etiology can be attributed to stress.

### BACKGROUND OF THE INVENTION

Many disease processes eventually result in tissue damage which in turn causes the release of tissue-specific markers into an individual's bodily fluid, e.g. the bloodstream, lymphatic system, cerebrospinal fluid and the like. A variety of diagnostic tests exist for checking abnormal levels of various constituents in bodily fluids. For example, urinalysis can check for abnormal levels of protein in the urine, and immunoassay tests for certain proteins are known for providing definitive diagnostic evidence of a particular disease state, e.g. heart attack, as set forth in U.S. Pat. Nos. 5,604,105, 5,744,358, 5,290,678, and 5,710,008, to the present inventor and incorporated by reference herein.

It is further well-known that the body's response to stress is highly complex. Virtually all of the body's systems, including the heart and blood vessels, the immune system, the lungs, the digestive system, the sensory organs, and the brain, are modified to meet a perceived danger or threat. Under normal situations, after the perceived threat has passed, the response becomes inactivated and levels of stress hormones return to normal, a condition called the relaxation response. Daily living brings with it an assortment of stress factors which can be short-term (acute) or long-term (chronic).

When presented with an immediate threat, an acute stress reaction, commonly known as the fight or flight response, is manifested. A portion of the brain known as the hypothalamic-pituitary-adrenal (HPA) system is activated, triggering the production and release of steroid hormones (glucocorticoids), including the primary stress hormone cortisol. Cortisol is a primary component in the body's initiation of response mechanisms involving, but not limited to the heart, lungs, circulation, metabolism, immune systems, and skin, which are required to deal quickly with the perceived danger or threat. Certain neurotransmitters (chemical messengers) called catecholamines, particularly those known as dopamine, norepinephrine, and epinephrine/adrenaline are released as a response to stress.

Alternatively, various chronic and on-going stressful situations that are not short-lived instigate similar bodily responses, after which the urge to act (e.g. to fight or to flee) must be suppressed. Some of the more common instigators of chronic stress include, but are not limited to on-going highly pressured work, problems regarding personal relationships, loneliness, worries with respect to financial problems, and the like.

As a result of the body's response to stress, heart rate and blood pressure increase, coupled with rapid breathing and increased oxygen intake, increased blood flow, and a discharge of red and white blood cells from the spleen, allowing the blood to transport more oxygen.

Simultaneously, the immune system mounts a similar defense to potentially critical areas. Parts of the immune system's functions are modified so that specific immune factors are altered. Fluids are diverted from nonessential locations, including the mouth, causing dryness and difficulty in talking and/or swallowing.

Additionally, blood flow is diverted away from the skin to aid in supporting the heart and muscle tissues. Lastly, stress shuts down digestive activity, a nonessential body function during short-term periods of physical exertion or crisis.

The above-noted physical changes in response to stress are an essential adaptation for meeting actual threats. However, these same changes, when translated into our modern lifestyle, can manifest themselves in a low, albeit a long-lasting, level of stress being placed upon all bodily systems, leading to a condition of chronic stress. It is theorized that these chronic stress conditions may produce physical or psychological damage over time.

Acute stress can also be harmful in certain situations. For example, stress can be a major trigger for angina, and incidents of acute stress have been associated with a higher risk for serious cardiac events, such as heart rhythm abnormalities and heart attacks, and even death from such events in people with heart disease. Stress may negatively affect the heart in several ways, e.g. by increasing the pumping action and rate of the heart and causing the constriction of arteries; causing a risk of reduced blood flow to the heart muscle; by creating an alteration of heart rhythms, and by causing increased platelet aggregation, which causes blood to become stickier, thereby increasing the likelihood of blood clot formation. Stress may also signal the body to release fat into the bloodstream, resulting in at least a temporary rise in blood-cholesterol levels, and possibly increased levels of homocysteine in the blood, a factor now strongly associated with heart disease.

In women, chronic stress may reduce estrogen levels, which are important for cardiac health. Stressful events may cause both men and women who have relatively low levels of serotonin (a neurotransmitter) to produce an increased level of certain immune system proteins, known as cytokines, which in high amounts cause inflammation and damage to cells, including possibly heart cells. Additional physical conditions exacerbated by stress include sudden and temporary increases in blood pressure, the long-term effects of which are not completely known, although over time multiple events of high blood pressure are implicated in injuries to the inner linings (the endothelium) of the blood vessels.

Chronic stress has also been associated with the development of insulin-resistance, a condition in which the body is unable to use insulin effectively to regulate glucose (blood sugar). Insulin-resistance is a primary factor in diabetes, particularly Type II or non insulin dependent diabetes (NIDDM). Stress can also exacerbate existing diabetes by impairing the body's ability to manage the disease effectively.

Stress also has been shown to have a significant effect on the brain; manifesting in loss of memory and an inability to concentrate. In severe situations, stress may even break down the blood-brain barrier, a physiological mechanism that helps protect the brain from toxins, bacteria, and the like harmful substances which might be transported by the bloodstream. It is theorized that prolonged cortisol exposure may actually cause damage to brain cells in the hippocampus, which may be irreversible.

What is lacking in the art is 1) a defined methodology for identifying unique stress mediated circulating markers indicative of identifiable precursor conditions, and 2) an ability to further said methodology by establishing a linking mechanism to correlate the presence of said circulating markers with the appearance of auto-antibodies specific thereto, thereby providing a mechanism for predicting the development and validating the presence of a particular disease state or precursor thereto.

### DESCRIPTION OF THE PRIOR ART

U.S. Pat. No. 6,140,067 relates to improved diagnostic methods for early detection of a risk for developing type 2 diabetes mellitus in humans, and screening assays for therapeutic agents useful in the treatment of type 2 diabetes mellitus, by comparing the levels of one or more indicators of altered mitochondrial function. Indicators of altered mitochondrial function include enzymes such as mitochondrial enzymes and ATP biosynthesis factors. Other indicators of altered mitochondrial function include mitochondrial mass, mitochondrial number and mitochondrial DNA content, cellular responses to elevated intracellular calcium and to apoptogens, and free radical production. Methods of treating, and of stratifying, human patients as such methods relate to disclosed indicators of altered mitochondrial function are also provided.

WO 00/65340 discloses a process for the discovery of novel protein-protein interactions in mammalian physiological pathways, including physiological disorders or diseases.

U.S. Pat. No. 5,010,175 teaches a method for obtaining and producing selected peptides with selected properties. U.S. Pat. No. 6,020,208 utilizes a general category of probe elements (i.e., sample presenting means) with Surfaces Enhanced for Laser Desorption/Ionization (SELDI), within which there are three (3) separate subcategories. The SELDI process is directed toward a sample presenting means (i.e., probe element surface) with surface-associated (or surface-bound) molecules to promote the attachment (tethering or anchoring) and subsequent detachment of tethered analyte molecules in a light-dependent manner, wherein the said surface molecule(s) are selected from the group consisting of photoactive (photolabile) molecules that participate in the binding (docking, tethering, or crosslinking) of the analyte molecules to the sample presenting means (by covalent attachment mechanisms or otherwise).

PCT/EP/04396 teaches a process for determining the status of an organism by peptide measurement. The reference teaches the measurement of peptides in a sample of the organism which contains both high and low molecular weight peptides and acts as an indicator of the organism's status. The reference concentrates on the measurement of low molecular weight peptides, i.e. below 30,000 Daltons, whose distribution serves as a representative cross-section of defined controls. Contrary to the methodology of the instant invention, the '396 patent strives to determine the status of a healthy organism, i.e. a “normal” and then use this as a reference to differentiate disease states. The present inventors do not attempt to develop a reference “normal”, but rather strive to specify particular markers which are evidentiary of at least one specific disease state, whereby the presence of said marker serves as a positive indicator of disease. This leads to a simple method of analysis which can easily be performed by an untrained individual, since there is a positive correlation of data. On the contrary, the '396 patent requires a complicated analysis by a highly trained individual to determine disease state versus the perception of non-disease or normal physiology.

Richter et al, Journal of Chromatography B, 726(1999) 25-35, refer to a database established from human hemofiltrate comprised of a mass database and a sequence database. The goal of Richter et al was to analyze the composition of the peptide fraction in human blood. Using MALDI-TOF, over 20,000 molecular masses were detected representing an estimated 5,000 different peptides. The conclusion of the study was that the hemofiltrate (HF) represented the peptide composition of plasma. No correlation of peptides with relation to normal and/or disease states is made.

The prior art fails to define a methodology for identifying unique stress mediated circulating markers indicative of identifiable precursor conditions, and 2) an ability to further said methodology by establishing a linking mechanism to correlate the presence of said circulating markers with the appearance of auto-antibodies specific thereto.

### SUMMARY OF THE INVENTION

Disease processes often result in tissue damage and the release of tissue-specific markers into the bodily fluids of an individual. This leads to the synthesis of antibodies against these “novel” proteins (since they are not normally present in the serum). The appearance of these antibodies and/or these so-called “novel” protein markers may be indicative of prior episodes or alternatively markers of disease progression. Furthermore, these markers as well as their antibodies may be used to monitor the response of a particular individual to therapeutic agents.

As a result of enduring sufficient amounts of stress, damage to various of the bodies organs begins to occur at a cellular or even a sub-cellular level. Such damage can be characterized as either reversible or irreversible. When such damage occurs, protein markers are release into the circulation, causing the immune system to become activated, in that these “new” protein markers are viewed as invading pathogens or foreign bodies whose threat must be neutralized. In an effort to persevere against this perceived threat, auto-antibodies are formed which are specific to these protein markers. These auto-antibodies can be characterized as sequela which are indicative of the original damaging insult to the organism (as defined herein an organism can be any plant or animal species). Their presence validates the theory that cellular damage acts as an initiator of an immune response leading to a cascade of auto-antibody production which ultimately manifests itself in a characteristic and often predictable disease state. Within the context of this invention, disease state will be defined as evidence of an abnormal physiological response, which response may be diagnostic of a specific disease or predictive of the development of a specific disease.

Accordingly, it is an objective of the instant invention to provide a biochemically-based methodology for ascertaining the presence and/or verifying the historical release of biopolymers, which have been shown to be indicative of a disease state or are predictive of the development of said disease state.

It is a further objective of the instant invention to isolate and identify specific biopolymer sequences, salts, isomers and fragments thereof, inclusive of polynucleotides encoding said biopolymers, oligonucleotides encoding said biopolymers, vectors comprising said polynucleotide or oligonucleotide, transformed or transfected host cells and hybridization probes inclusive thereof, which are indicative of a particular disease state.

It is yet another objective of the instant invention to provide immunoassay tests which utilize specific biopolymers for formulating 1)diagnostic tests for characterizing a disease state and 2)risk assessment tests predictive of the development of a disease state.

It is a still further objective of the invention to provide pre-clinical animal models including knock-in, knockout, conditional knock-out and animals expressing modulated expression of markers, said animals being useful for studying specific disease states and therapeutic solutions thereto.

Other objects and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objects and features thereof.

### BRIEF DESCRIPTION OF THE FIGURES

FIG. 1 is a block diagram illustrative of the methodology for disease state analysis, progression and confirmation.

FIG. 2 is one embodiment of proteomics discovery specific to heart failure;

FIG. 3 is a second embodiment of proteomics discovery specific to heart failure;

FIG. 4 is a series of spectra depicting particular markers educed from the discovery procedure related to congestive heart failure.

### DETAILED DESCRIPTION OF THE INVENTION

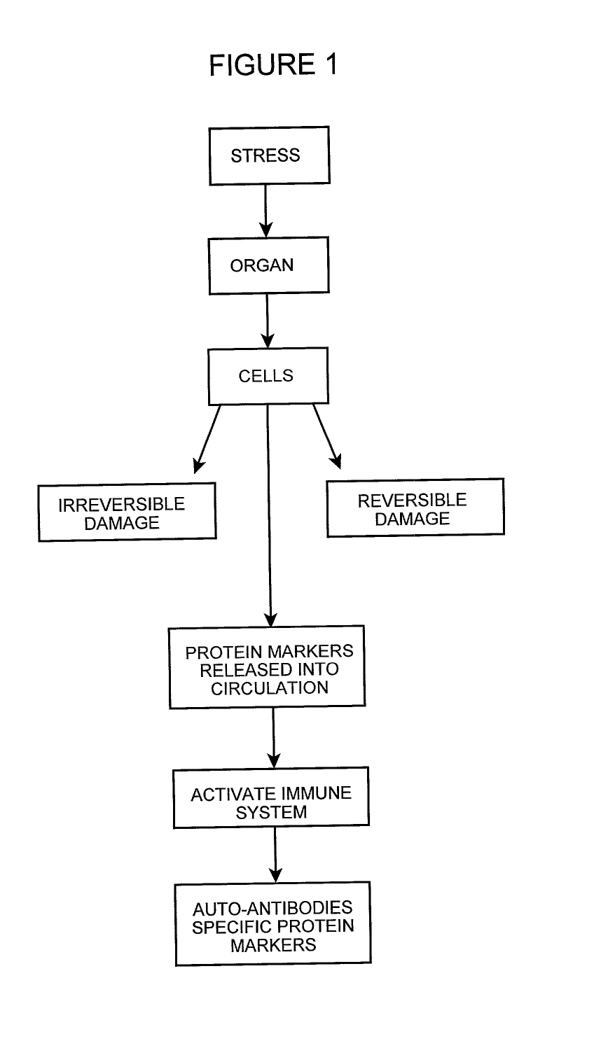

Referring now to FIG. 1, the initiating event in the genesis of a disease state is considered to be the effect of stress on an individual's organ(s). An organ is defined as a differentiated part or member having a specific function in a plant or animal. When an individual is subjected to various forms of stress, inclusive of acute and chronic stress, and particularly to those forms which manifest themselves in creating atypical physiological conditions for various organ systems, damage begins to occur at a cellular or sub-cellular level. As indicated previously, such conditions as high blood pressure, volume overload, ischemic episodes and the like, initiate a sequence of cell damaging events. Initially, these events may only cause damage to the outer membranes of cells, causing a sloughing off of portions of the exterior cellular matrices, which can be broadly defined as reversible damage. As the length of time and/or severity of the stress inducing condition increases, the outer membranous regions of the cells begin to break down, resulting in membrane rupture and release of intra-cellular components, broadly defined as irreversible cell damage. As cellular damage evolves and increases, specific biopolymer markers, which are illustrated by, but not limited to particular polypeptides, peptides, immunologically detectable fragments and isomers thereof (e.g. by enzyme activity from proteolytic breakdown), and auto-antibodies thereto, are released into the individual's bodily fluids. The particular protein markers are highly tissue specific and are amenable to isolation for the development of both diagnostic and risk-assessment tests, e.g. rapid point-of-care format (POC) immunoassay tests and microtitration format, in accordance with known methods, thereby providing a tool for determining their presence in the circulating sera tested. Any suitable immunoassay method may be used, some non-limiting examples being sandwich enzyme-linked immunoassays (ELISA), radioimmunoassays, competitive binding assays, homogeneous assays, heterogeneous, direct and indirect labeled assays, and double antibody techniques, etc. as known in the art and as disclosed by the instant inventor in U.S. Pat. No. 5,747,274, the contents of which are herein incorporated by reference. These immunoassays may be quantitative or semiquantitative such that they include the ability to discriminate between a level which is elevated a statistically significant level above the norm, e.g. about two standard deviations above normal, and a level which is not above that threshold. For purposes of this invention, the bodily fluids tested, which will be referred to as sera, will include, but are not limited to, blood and blood products e.g. plasma, serum, cytolyzed blood (e.g. blood treated with hypotonic buffer or detergents), lymphatic fluid, cerebrospinal fluid, pericardial fluid, vitreous humor, and the like.

As these markers are released, the effected individual's immune system views them as “novel” proteins, since their presence in the circulating sera is outside of the norm. Activation of the immune system to these “novel” proteins causes an immune response resulting in the production of auto-antibodies. The instant invention recognizes the presence of these auto-antibodies as having far-reaching significance. Firstly, the development of auto-antibodies to specific protein markers evidences an immune response to the markers themselves and validates their presence at some point in time, even if they are no longer present. Secondly, the presence and rate of production of specific auto-antibodies are predictive of the development of a particular disease state, and can provide valuable insight into the evolution and predicted severity of said disease state.

Although not wishing to be limited to any particular disease or diagnostic methodology, the protocol for assessment of disease progression and confirmation will now be characterized as it relates to the continuum of interrelated diseases known as Syndrome-X.

Syndrome-X is a multifaceted syndrome, which occurs frequently in the general population. A large segment of the adult population of industrialized countries develops this metabolic syndrome, produced by genetic, hormonal and lifestyle factors such as obesity, physical inactivity and certain nutrient excesses. This disease is characterized by the clustering of insulin resistance and hyperinsulinemia, and is often associated with dyslipidemia (atherogenic plasma lipid profile), essential hypertension, abdominal (visceral) obesity, glucose intolerance or noninsulin-dependent diabetes mellitus and an increased risk of cardiovascular events. Abnormalities of blood coagulation (higher plasminogen activator inhibitor type I and fibrinogen levels), hyperuricemia and microalbuminuria have also been found in metabolic syndrome-X.

The instant inventors view the Syndrome-X continuum in its cardiovascular light, while acknowledging its important metabolic component. The first stage of Syndrome X consists of insulin resistance, abnormal blood lipids (cholesterol and triglycerides), obesity, and high blood pressure (hypertension). Any one of these four first stage conditions signals the start of Syndrome X, and is characterized as a stressful condition placed upon the organism, whose reaction to this stressful condition can manifest itself as Syndrome X Hale & Slavin, P.A. - 2132.011 -16and diseases related thereto.

Each first stage Syndrome X condition risks leading to another. For example, increased insulin production is associated with high blood fat levels, high blood pressure, and obesity. Furthermore, the effects of the first stage conditions are additive; an increase in the number of conditions causes an increase in the risk of developing more serious diseases on the Syndrome X continuum.

A patient who begins the Syndrome X continuum risks spiraling into a maze of increasingly deadly diseases. The next stages of the Syndrome X continuum lead to overt diabetes, kidney failure, and heart failure, with the possibility of stroke and heart attack at any time. Syndrome X is a dangerous continuum, and preventative medicine is the best defense. Diseases are currently most easily diagnosed in their later stages, but controlling them at a late stage is extremely difficult. Disease prevention is much more effective at an earlier stage.

By utilizing the strategy as outlined in FIG. 1, risk-assessment tests are developed which allow physicians to identify asymptomatic patients before they suffer an irreversible event such as diabetes, kidney failure, and heart failure, and enable effective disease management and preventative medicine. Additionally, the specific diagnostic tests which evolve from this methodology provide a tool for rapidly and accurately diagnosing acute Syndrome X events such as heart attack and stroke, and facilitate treatment.

Protein analysis can be carried out by a number of different methods, including but not limited to 1D, 2D gel electrophoresis characterization, Western blotting, liquid chromatography quadropole ion trap electrospray (LCQ-MS), Matrix Assisted Laser Desorption Ionization/Time of Flight (MALDI-TOF), and Surface Enhanced Laser Desorption Ionization Spectroscopy/Time of Flight (SELDI/TOF).

In the particular examples listed below, SELDI/TOF was the preferred form of biopolymer detection.

### EXAMPLES

Serum samples from individuals were analyzed using Surface Enhanced Laser Desorption Ionization (SELDI) as a proteomic investigative technique using the Ciphergen PROTEINCHIP system. The chip surfaces included, but were not limited to IMAC-3-Ni, SAX2 surface chemistries, gold chips, and the like.

Preparatory to the conduction of the SELDI MS procedure, various preparatory steps were carried out in order to maximize the diversity of discernible moities educable from the sample. Utilizing a type of micro-chromatographic column called a C18-ZIPTIP available from the Millipore company, the following preparatory steps were conducted.

1. Dilute sera in sample buffer

2. Aspirate and dispense ZIP TIP in 50% Acetonitrile

3. Aspirate and dispense ZIP TIP in Equilibration solution

4. Aspirate and Dispense in serum sample

5. Aspirate and Dispense ZIP TIP in Wash solution

6. Aspirate and Dispense ZIP TIP in Elution Solution

Illustrative of the various buffering compositions useful in the present invention are:

Sample Buffers (various low pH's): Hydrochloric acid (HCl),

Formic acid, Trifluoroacetic acid (TFA),

Equilibration Buffers (various low pH's): HCl, Formic acid, TFA;

Wash Buffers (various low pH's): HCl, Formic acid, TFA;

Elution Solutions (various low pH's and % Solvents): HCl, Formic acid, TFA;

Solvents: Ethanol,Methanol, Acetonitrile.

Spotting was then performed, for example upon a Gold Chip in the following manner:

1. spot 2 ul of sample onto each spot

2. let sample partially dry

3. spot 1 ul of matrx, and let air dry.

HiQ Anion Exchange Mini Column Protocol

1. Dilute sera in sample/running buffer;

2. Add HiQ resin to column and remove any air bubbles;

3. Add Uf water to aid in column packing;

1. Add sample/running buffer to equilibrate column;

2. Add diluted sera;

3. Collect all the flow through fraction in Eppendorf tubes until level is at resin;

4. Add sample/running buffer to wash column;

5. Add elusion buffer and collect elusion in Eppendorf tubes.

Illustrative of the various buffering compositions useful in this technique are:

Sample/Running buffers: including but not limited to Bicine buffers of various molarities, pH's, NaCl content, Bis-Tris buffers of various molarities, pH's, NaCl content, Diethanolamine of various molarities, pH's, NaCl content, Diethylamine of various molarities, pH's, NaCl content, Imidazole of various molarities, pH's, NaCl content, Tricine of various molarities, pH's, NaCl content, Triethanolamine of various molarities, pH's, NaCl content, Tris of various molarities, pH's, NaCl content.

Elution Buffer: Acetic acid of various molarities, pH's, NaCl content, Citric acid of various molarities, pH's, NaCl content, HEPES of various molarities, pH's, NaCl content, MES of various molarities, pH's, NaCl content, MOPS of various molarities, pH's, NaCl content, PIPES of various molarities, pH's, NaCl content, Lactic acid of various molarities, pH's, NaCl content, Phosphate of various molarities, pH's, NaCl content, Tricine of various molarities, pH's, NaCl content.

Chelating Sepharose Mini Column

1. Dilute Sera in Sample/Running buffer;

2. Add Chelating Sepharose slurry to column and allow column to pack;

3. Add UF water to the column to aid in packing;

4. Add Charging Buffer once water is at the level of the resin surface;

5. Add UF water to wash through non bound metal ions once charge buffer washes through;

6. Add running buffer to equilibrate column for sample loading;

7. Add diluted serum sample;

8. Add running buffer to wash unbound protein;

9. Add elution buffer and collect elution fractions for analysis;

10. Acidify each elution fraction.

Illustrative of the various buffering compositions useful in this technique are:

Sample/Running buffers including but not limited to Sodium Phosphate buffers at various molarities and pH's;

Charging buffers including but not limited to Nickel Chloride, Nickel Sulphate, Copper II Chloride, Zinc Chloride or any suitable metal ion solution;

Elution Buffers including but not limited to Sodium phosphate buffers at various molarities and pH's containing various molarities of EDTA and/or Imidazole.

HiS Cation Exchange Mini Column Protocol

1. Dilute sera in sample/running buffer;

2. Add HiS resin to column and remove any air bubbles;

3. Add Uf water to aid in column packing;

4. Add sample/running buffer to equilibrate column for sample loading;

5. Add diluted sera to column;

6. Collect all flow through fractions in Eppendorf tubes until level is at resin.

7. Add sample/running buffer to wash column.

8. Add elusion buffer and collect elusion in Eppendorf tubes.

Illustrative of the various buffering compositions useful in this technique are:

Sample/Running buffers: including but not limited to Bicine buffers of various molarities, pH's, NaCl content, Bis-Tris buffers of various molarities, pH's, NaCl content, Diethanolamine of various molarities, pH's, NaCl content, Diethylamine of various molarities, pH's, NaCl content, Imidazole of various molarities, pH's, NaCl content, Tricine of various molarities, pH's, NaCl content, Triethanolamine of various molarities, pH's, NaCl content, Tris of various molarities, pH's, NaCl content.

Elution Buffer: Acetic acid of various molarities, pH's, NaCl content, Citric acid of various molarities, pH's, NaCl content, HEPES of various molarities, pH's, NaCl content, MES of various molarities, pH's, NaCl content, MOPS of various molarities, pH's, NaCl content, PIPES of various molarities, pH's, NaCl content, Lactic acid of various molarities, pH's, NaCl content, Phosphate of various molarities, pH's, NaCl content, Tricine of various molarities, pH's, NaCl content.

The procedure for profiling serum samples is described below:

Following the preparatory steps illustrated above, various methods for use of the PROTEINCHIP arrays, available for purchase from Ciphergen Biosystems (Palo Alto, Calif.), may be practiced. Illustrative of one such method is as follows. The first step involved treatment of each spot with 20 ml of a solution of 0.5 M EDTA for 5 minutes at room temperature in order to remove any contaminating divalent metal ions from the surface. This was followed by rinsing under a stream of ultra-filtered, deionized water to remove the EDTA. The rinsed surfaces were treated with 20 ml of 100 mM Nickel sulfate solution for 5 minutes at room temperature after which the surface was rinsed under a stream of ultrafiltered, deionized water and allowed to air dry.

Serum samples (2 ml) were applied to each spot (now “charged” with the metal-Nickel) and the PROTEINCHIP was returned to the plastic container in which it was supplied. A piece of moist KIMWIPE was placed at the bottom of the container to generate a humid atmosphere. The cap on the plastic tube was replaced and the chip allowed to incubate at room temperature for one hour. At the end of the incubation period, the chip was removed from the humid container and washed under a stream of ultra-filtered, deionized water and allowed to air dry. The chip surfaces (spots) were now treated with an energy-absorbing molecule that helps in the ionization of the proteins adhering to the spots for analysis by Mass Spectrometry. The energy-absorbing molecule in this case was sinapinic acid and a saturated solution prepared in 50% acetonitrile and 0.05% TFA was applied (1 ml) to each spot. The solution was allowed to air dry and the chip analyzed immediately using MS (SELDI).

Serum samples from patients suffering from a variety of disease states were analyzed using one or more protein chip surfaces, e.g. a gold chip or an IMAC nickel chip surface as described above and the profiles were analyzed to discern notable sequences which were deemed in some way evidentiary of at least physiological condition or disease state.

Patient specific samples were obtained and the data used to formulate a library of proteomic materials having characteristics identifiable with both normal and abnormal physiological conditions or predictive hallmarks thereof.

In order to purify the disease specific marker and further characterize the sequence thereof, additional processing was performed.

For example, Serum (20 ml) was (diluted 5-fold with phosphate buffered saline) concentrated by centrifugation through a YM3 MICROCON spin filter (Amicon) for 20 min at 10,000 RPM at 4° C. in a Beckman MICROCENTRIFuge R model bench top centrifuge. The filtrate was discarded and the retained solution, which contained the two peptides of interest, was analyzed further by tandem mass spectrometry to deduce their amino acid sequences. Tandem mass spectrometry was performed at the University of Manitoba's (Winnipeg, Manitoba, Canada) mass spectrometry laboratory using the procedures that are well known to practitioners of the art.

In order to more clearly show the effects of stress with regard to disease, the use of the proteomics discovery process is illustrated by, but not limited to congestive heart failure.

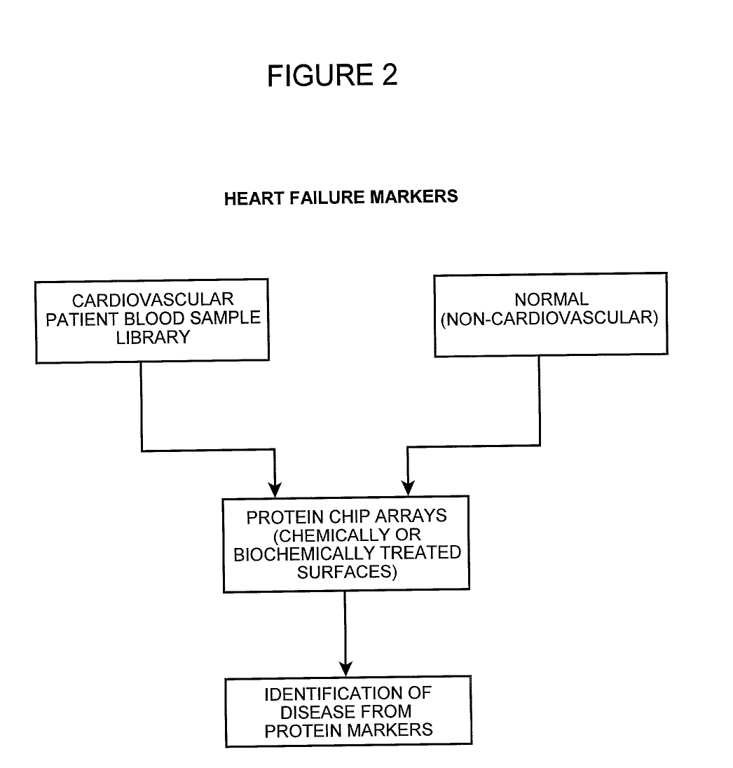

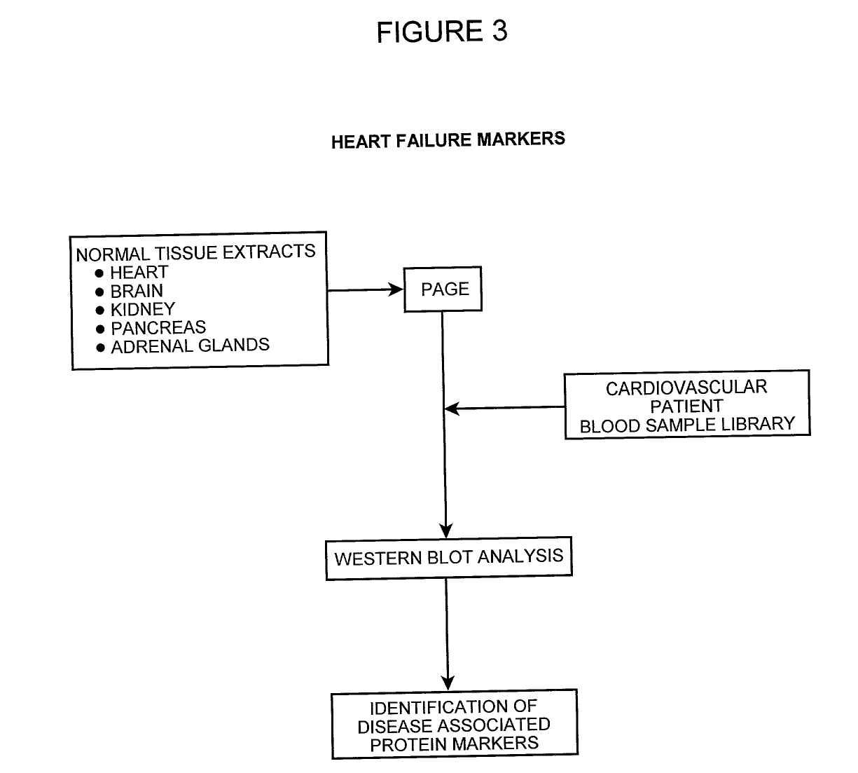

FIGS. 2 and 3 are illustrative of the procedure outlined above wherein the results are specific to heart failure.

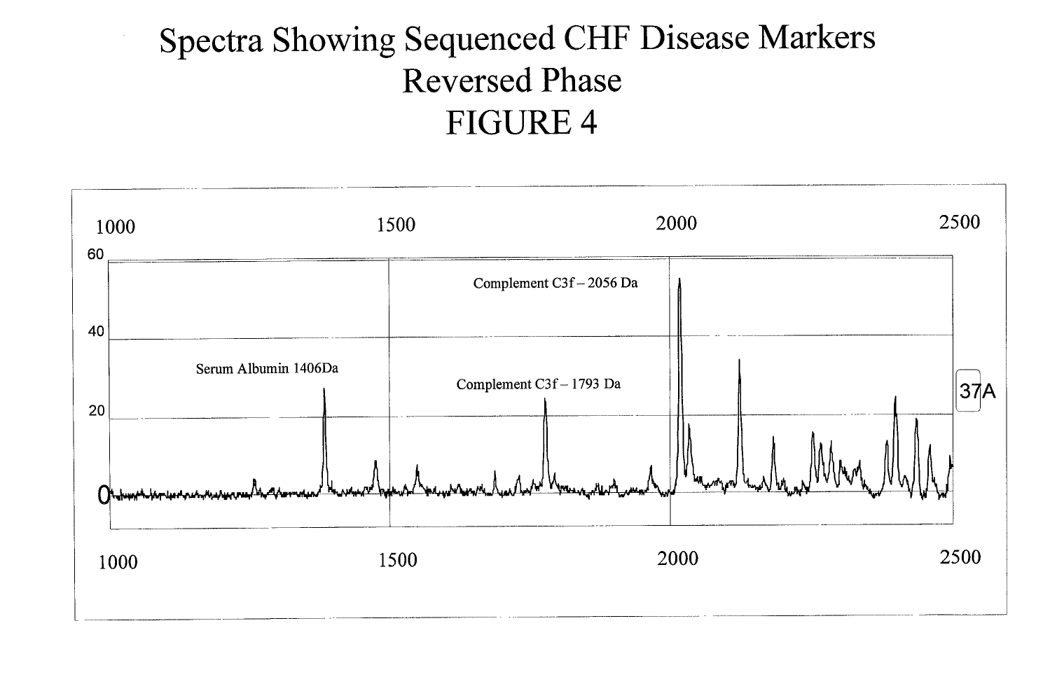

FIG. 4 particularly sets forth exemplary, but non-limiting markers deemed to be indicative of heart failure.

In accordance with various stated objectives of the invention, the skilled artisan, in possession of the specifically isolated proteomic material, would readily carry out known techniques in order to raise purified biochemical materials, e.g. monoclonal and/or polyclonal antibodies, which are useful in the production of methods and devices useful as point-of-care rapid assay diagnostic or risk assessment devices as are known in the art.

The specific proteomic materials which are analyzed according to the method of the invention are released into the circulation and may be present in the blood or in any blood product, for example plasma, serum, cytolyzed blood, e.g. by treatment with hypotonic buffer or detergents and dilutions and preparations thereof, and other body fluids, e.g. CSF, saliva, urine, lymph, and the like. The presence of each proteomic material marker, or alternatively an autoantibody which validates the autoimmune process raised by the organism against a particular marker, is determined using antibodies specific for each of the markers and detecting specific binding of each antibody to its respective marker. Any suitable direct or indirect assay method may be used to determine the level of each of the specific markers measured according to the invention. The assays may be competitive assays, sandwich assays, and the label may be selected from the group of well-known labels such as radioimmunoassay, fluorescent or chemiluminescence immunoassay, or immunoPCR technology. Extensive discussion of the known immunoassay techniques is not required here since these are known to those of skilled in the art. See Takahashi et al. (Clin Chem 1999;45(8):1307) for S100B assay.

A monoclonal antibody specific against the proteomic material sequence isolated by the present invention may be produced, for example, by the polyethylene glycol (PEG) mediated cell fusion method, in a manner well-known in the art.

Traditionally, monoclonal antibodies have been made according to fundamental principles laid down by Kohler and Milstein. Mice are immunized with antigens, with or without, adjuvants. The splenocytes are harvested from the spleen for fusion with immortalized hybridoma partners. These are seeded into microtitre plates where they can secrete antibodies into the supernatant that is used for cell culture. To select from the hybridomas that have been plated for the ones that produce antibodies of interest the hybridoma supernatants are usually tested for antibody binding to antigens in an ELISA (enzyme linked immunosorbent assay) assay. The idea is that the wells that contain the hybridoma of interest will contain antibodies that will bind most avidly to the test antigen, usually the immunizing antigen. These wells are then subcloned in limiting dilution fashion to produce monoclonal hybridomas. The selection for the clones of interest is repeated using an ELISA assay to test for antibody binding. Therefore, the principle that has been propagated is that in the production of monoclonal antibodies the hybridomas that produce the most avidly binding antibodies are the ones that are selected from among all the hybridomas that were initially produced. That is to say, the preferred antibody is the one with highest affinity for the antigen of interest.

There have been many modifications of this procedure such as using whole cells for immunization. In this method, instead of using purified antigens, entire cells are used for immunization. Another modification is the use of cellular ELISA for screening. In this method instead of using purified antigens as the target in the ELISA, fixed cells are used. In addition to ELISA tests, complement mediated cytotoxicity assays have also been used in the screening process. However, antibody-binding assays were used in conjunction with cytotoxicity tests. Thus, despite many modifications, the process of producing monoclonal antibodies relies on antibody binding to the test antigen as an endpoint.

The purified monoclonal antibody is utilized for immunochemical studies.

Polyclonal antibody production and purification utilizing one or more animal hosts in a manner well-known in the art can be performed by a skilled artisan.

Another objective of the present invention is to provide reagents for use in diagnostic assays for the detection of the particularly isolated proteomic materials of the present invention.

In one mode of this embodiment, the proteomic materials, e.g. the disease specific marker sequences of the present invention may be used as antigens in immunoassays for the detection of those individuals suffering from the disease known to be evidenced by said marker sequence. Such assays may include but are not limited to: radioimmunoassay, enzyme-linked immunosorbent assay (ELISA), “sandwich” assays, precipitin reactions, gel diffusion immunodiffusion assay, agglutination assay, fluorescent immunoassays, protein A or G immunoassays and immunoelectrophoresis assays.

According to the present invention, monoclonal or polyclonal antibodies produced against the isolated proteomic materials of the instant invention are useful in an immunoassay on samples of blood or blood products such as serum, plasma or the like, spinal fluid or other body fluid, e.g. saliva, urine, lymph, and the like, to diagnose patients with the characteristic disease state linked to said marker sequence. The antibodies can be used in any type of immunoassay. This includes both the two-site sandwich assay and the single site immunoassay of the non-competitive type, as well as in traditional competitive binding assays.

Particularly preferred, for ease and simplicity of detection, and its quantitative nature, is the sandwich or double antibody assay of which a number of variations exist, all of which are contemplated by the present invention. For example, in a typical sandwich assay, unlabeled antibody is immobilized on a solid phase, e.g. microtiter plate, and the sample to be tested is added. After a certain period of incubation to allow formation of an antibody-antigen complex, a second antibody, labeled with a reporter molecule capable of inducing a detectable signal, is added and incubation is continued to allow sufficient time for binding with the antigen at a different site, resulting with a formation of a complex of antibody-antigen-labeled antibody. The presence of the antigen is determined by observation of a signal which may be quantitated by comparison with control samples containing known amounts of antigen.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and drawings/figures.

One skilled in the art will readily appreciate that the present invention is well adapted to carry out the objectives and obtain the ends and advantages mentioned, as well as those inherent therein. The oligonucleotides, peptides, polypeptides, biologically related compounds, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. An analytical method for assessing a disease state comprising:
obtaining a sera sample from an organism;
analyzing said sera sample to identify at least one biopolymer contained therein;
referencing said at least one identified biopolymer against a biopolymer library adapted to characterize said identified biopolymer as being indicative of a particular disease state; and
specifying said particular disease state.
